(19) United States
(12) Patent Application Publication
(10) Pub. No.: US 2015/0173591 A1
(43) Pub. Date: Jun. 25, 2015

(54) DEVICE OF ANTI-FOGGING ENDOSCOPE SYSTEM AND ITS METHOD

(71) Applicant: Qingdao Novelbeam Technology Co., LTD., Qingdao (CN)

(72) Inventors: Yao Zheng, Qingdao (CN); Changming Gu, Qingdao (CN); Anmin Zheng, Qingdao (CN); Rongzhuang Mao, Qingdao (CN); Chunxin Yang, Qingdao (CN)

(21) Appl. No.: 14/379,269

(22) PCT Filed: Jul. 23, 2013
(86) PCT No.: PCT/CN2013/079898

§ 371 Date: Aug. 15, 2014

(30) Foreign Application Priority Data
Sep. 5, 2012 (CN) 201210324982.0

(51) Int. Cl. A61B 1/00; A61B 1/07; A61B 1/06

(52) U.S. Cl. CPC A61B 1/00096; A61B 1/0684; A61B 1/07

(57) ABSTRACT

The invention relates to a device of anti-fogging endoscope system and its method, falling in the minimally invasive medical technical field, wherein a near-infrared lighting source for anti-fogging is added on the basis of the traditional endoscope system, a beam of which is coupled into the lighting transmission path in color combination, and changes material properties of a distal optical window plate to transmit the visible light, ensuring that surgical filed is lighted by white light, while the temperature of the distal optical window plate is elevated by absorbing energy of the near-infrared light to reduce the temperature difference between the distal optical window plate of endoscope and a human body, to realize the purpose of anti-fogging. Comparing the invention with anti-fogging ways of preheating with physiological saline, smearing anti-fogging oil, and so on, as to the doctor's performances, the invention has the more convenient and easier advantages, and there is not a latent risk of incompletely disinfecting when using physiological saline, anti-fogging oil and so on, which is suitable to be used in various endoscopic operations, and is specially suitable to be used in the endoscopic operations wherein the endoscope needs to be repeatedly inserted into the human body and drawn out from the human body, in order to play the effect of anti-fogging.

### TECHNICAL FIELD OF THE INVENTION

The invention relates to a device of anti-fogging endoscope system and its method, falling in the minimally invasive medical technical field.

### BACKGROUND OF THE INVENTION

Medical endoscopes have been widely used in the minimally invasive surgery, however, the current minimally invasive surgery using an endoscope system has an operating deficiency, i.e., when an endoscope is working in a human body, due to a temperature difference between the internal and external of the human body, a part of the water vapor which contacts the endoscope having a lower temperature usually forms fog on a distal optical window plate of the endoscope to influence the imaging clarity of the surgical field, which blurs the image of surgical field showing on a displayer, and affects the observing and operating of a doctor. In order to remove the fogging on the endoscope, doctors mainly apply ways of using physiological saline to preheat endoscopes, smearing anti-fogging oil and so on, which have some effect on relieving the problem of fogging on the endoscope, while their defect is making the surgical preparation complex, there is a latent risk of incompletely disinfecting, and the time of surgical preparation is prolonged. Accordingly, the best way is directly providing an anti-fogging or defogging solution on an endoscope system.

### SUMMARY OF THE INVENTION

The invention aims to solve a technical problem of providing a device of anti-fogging endoscope system and its method, wherein a near-infrared lighting source for anti-fogging is added on the basis of the traditional endoscope system, a beam of which is coupled into the lighting transmission path in color combination, and changes material properties of a distal optical window plate to transmit the visible light, ensuring that surgical filed is lighted by white light, while the temperature of the distal optical window plate is elevated by absorbing energy of the near-infrared light to reduce the temperature difference between the distal optical window plate of endoscope and a human body, to realize the purpose of anti-fogging.

Now, combining with the accompanying figure, the technical solution of the invention is specified:

A device of anti-fogging endoscope system, including a lighting source 1, a transmission fiber 2, and an endoscope 3, wherein the lighting source 1 includes a lighting source of white light 11 and a coupling optical lens 14; the lighting source of white light 11 is a xenon lamp, a white light LED or a mixed white light LED of three primary colors of red, green, and blue, which provides a light output in visible light band; when the xenon lamp is used, the power falls in the range of 250 W˜350 W; when the white light LED is used, the power falls in the range of 50 W˜100 W; when the mixed white light LED of three primary colors of red, green, and blue is used, the power falls in the range of 150 W˜200 W; the coupling optical lens 14 is an optical lens or a set of optical lenses; the transmission fiber 2 is made by binding several fibers having the diameter of several tens of microns together, and is polished on its incident surface and exit surface; the focusing plane of coupling optical lens 14 and the incident surface of transmission fiber 2 are in coincidence; the endoscope 3 includes an endoscope optical lens system 31, a distal optical window plate 32, a light guide fiber bundle 33, an endoscope housing body 34, and a fiber connecting interface 35; the endoscope optical lens system 31 is made of several tens of optical lenses to image the surgical field; the endoscope housing body 34 is mainly made from stainless steel material, which is polished on its surface; the distal optical window plate 32 is a transparent optical material, and can transmit the visible light, which is located in front of the endoscope optical lens system 31, and is welded or adhered to the endoscope housing body 34; the light guide fiber bundle 33 is made by binding several fibers having the diameter of several tens of microns together, and is polished on its incident surface and exit surface; the incident surface of light guide fiber bundle 33 is located inside the fiber connecting interface 35; the incident surface of light guide fiber bundle 33 is connected to the exit surface of transmission fiber 2 in coupling via the fiber connecting interface 35; the light guide fiber bundle 33 is located in the annular space between the optical lens system 31 and the endoscope housing body 34, of which exit surface is against the distal optical window plate 32, which characterized in that, the lighting source 1 also includes a near-infrared lighting source for anti-fogging 12 and a color combination filter 13; the color combination filter 13 is a optical plate, which is located between the lighting source of white light 11 and the coupling optical lens 14 in way of having a intersection angle of 45° from the coupling optical lens 14; the color combination filter 13 is plated with an optical film which transmits the visible light and reflects the near-infrared light; the near-infrared lighting source for anti-fogging 12 is a light source which only emits the near-infrared light, such as a light source of semiconductor laser, a light source of pump laser, and so on, of which the wavelength range of the emitted light is 780 nm˜1100 nm, and the power of the emitted light in full power is between 5 watt and 10 watt; the near-infrared lighting source for anti-fogging 12 is set in way of being perpendicular to the lighting source of white light 11 and having a intersection angle of 45° from the color combination filter 13; the lighting source of white light 11 and the near-infrared lighting source for anti-fogging 12 can be independently controlled to turn on-off and adjust luminance; the distal optical window plate 32 of the endoscope 3 is made from an optical material improving its spectral characteristics, which not only can transmit the visible light, but also can absorb the near-infrared light, wherein the average transmissivity of the visible light is above 80%, and the average absorptivity of the near-infrared light is above 80%.

According to the above device of anti-fogging endoscope system, an anti-fogging method of the anti-fogging endoscope system including the following steps:

(1) 2˜3 minutes before an operation, the near-infrared lighting source for anti-fogging 12 is turned on in full power, near-infrared light transmits through the coupling optical lens 14, the transmission fiber 2, and the light guide fiber bundle 33 in order, and then irradiates on the distal optical window plate 32; the distal optical window plate 32 absorbs energy of the near-infrared light, and the temperature rises up.

(2) After 30 seconds˜1 minute, the temperature of the distal optical window plate 32 rises from room temperature of 25 up to about 37° C., then the power of the near-infrared lighting source for anti-fogging 12 is reduced to 10%˜30% of full power to keep the temperature of the distal optical window plate 32 around 37° C.

(3) Before the doctor inserts the endoscope into a human body, the near-infrared lighting source for anti-fogging 12 is turned off, the lighting source of white light 11 is turned on, and then the endoscope is inserted into the human body; due to the temperature of the distal optical window plate 32 is approximately consistent with the temperature of the human body, no fogging will happen, and so the doctor can perform the normal observation and operation, to achieve the purpose of anti-fogging.

The invention has the following positive improvements: no great change will be made on the basis of the traditional endoscope system, only a near-infrared lighting source for anti-fogging is added, and the material of the distal optical window plate is changed to achieve an anti-fogging function on system level. Comparing with anti-fogging ways of preheating with physiological saline, smearing anti-fogging oil, and so on, as to the doctor's performances, the invention has the more convenient and easier advantages, and there is not a latent risk of incompletely disinfecting when using physiological saline, anti-fogging oil and so on.

### BRIEF DESCRIPTION OF THE DRAWINGS

FIG. 1 is a structural schematic diagram of a device of anti-fogging endoscope system disclosed in the invention.

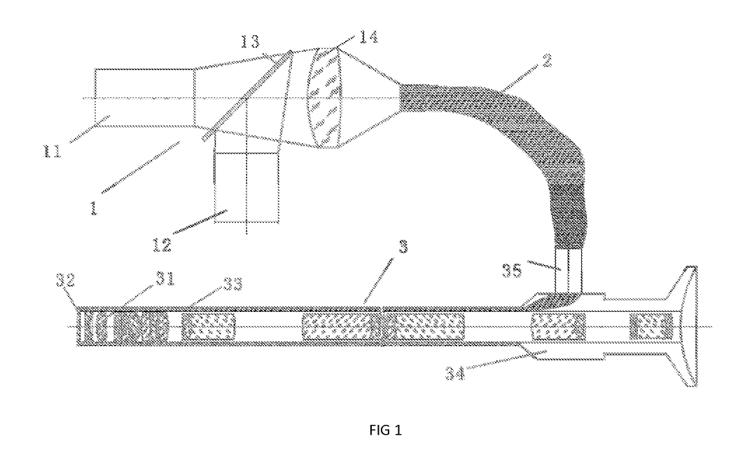

### DETAILED DESCRIPTION OF THE INVENTION

Now, the technical solution and working principle of the invention are specified by combining with the accompanying figure and embodiments.

All the embodiments have completely same structures and methods with that of the device and method described in the summary of the invention. In order to avoid a repetition, the following embodiments only present critical technical data.

### Embodiment 1

The lighting source of white light 11 is a white light LED having a total power of 80 W. The near-infrared lighting source for anti-fogging 12 is a semiconductor laser having a radiation wavelength of 808 nm and a full power output of 5 W. The distal optical window plate 32 has an absorptivity of 80% at 808 nm, and the average transmissivity of the visible light is 90%. The near-infrared lighting source for anti-fogging 12 is turned on in full power last 1 minute, the temperature of the distal optical window plate 32 rises from room temperature of 25° C. up to about 37° C., then the power is reduced to 30% of full power, keeping the temperature around 37° C.

The working principle is: 2˜3 minutes before an operation, the semiconductor laser of 808 nm is turned on to output a laser of 808 nm with 5 W. The laser of 808 nm passes through the color combination filter 13, and then is reflected into the coupling optical lens 14. The coupling optical lens 14 makes the near-infrared light of 808 nm focus and couple into the transmission fiber 2, and exit from the exit surface of the transmission fiber 2. The exited near-infrared light of 808 nm couples into the light guide fiber bundle 33 via the fiber connecting in 35, and then exit from the exit surface of the light guide fiber bundle 33. The exited near-infrared light of 808 nm irradiates onto the distal optical window plate 32, the near-infrared light of 80% is absorbed by the distal optical window plate, and then the temperature of the distal optical window plate 32 begins to rise up from the room temperature. After 1 minute, the temperature of distal optical window plate rises up to around 37° C. At this time, the power of the near-infrared lighting source for anti-fogging 12 is reduced to 30% of full power to keep the temperature of distal optical window plate around 37° C. Before the endoscope 3 is inserted into a human body, the near-infrared lighting source for anti-fogging 12 is turned off. Then the lighting source of white light 11 is turned on, and the white light emitted from the lighting source of white light 11 irradiates to the color combination filter 13, and passes through the color combination filter 13 into the coupling optical lens 14. The coupling optical lens 14 makes the white light focus and couple into the transmission fiber 2, and exit from the exit surface of the transmission fiber 2. The exited white light couples into the light guide fiber bundle 33 via the fiber connecting interface 35, and then exit from the exit surface of the light guide fiber bundle 33. The exited white light irradiates onto the distal optical window plate 32, and white light of about 90% transmits through the distal optical window plate 32 to light the surgical field. In this way, due to the temperature of the distal optical window plate 32 is approximately consistent with the temperature of the human body inside, no fogging will happen on the distal optical window plate 32, while the while light lights the surgical field to ensure the normal observation and operation performances in the surgical field.

### Embodiment 2

The lighting source of white light 11 is a mixed white light LED of three primary colors of red, green, and blue having a total power of 170 W. The near-infrared lighting source for anti-fogging 12 is a semiconductor laser having a radiation wavelength of 940 nm and a full power output of 10 W. The distal optical window plate 32 has an absorptivity of 90% at 940 nm, and the average transmissivity of the visible light is 85%. The near-infrared lighting source for anti-fogging 12 is turned on in full power last 30 seconds, the temperature of the distal optical window plate 32 rises from room temperature of 25° C. up to about 37° C., then the power is reduced to 10% of full power, keeping the temperature around 37° C.

### Embodiment 3

The lighting source of white light 11 is a xenon lamp having a total power of 350 W. The near-infrared lighting source for anti-fogging 12 is a Nd:YAG laser having a radiation wavelength of 1064 nm and a full power output of 7 W. The distal optical window plate 32 has an absorptivity of 95% at 1064 nm, and the average transmissivity of the visible light is 80%. The near-infrared lighting source for anti-fogging 12 is turned on in full power last 45 seconds, the temperature of the distal optical window plate 32 rises from room temperature of 25° C. up to about 37° C., then the power is reduced to 20% of full power, keeping the temperature around 37° C.

The working principles of embodies 2 and 3 is similar to that of embody 1, and so there is unnecessary to repeat.

The device of anti-fogging endoscope system and its method disclosed in the invention is suitable to be used in various endoscopic operations, and is specially suitable to be used in the endoscopic operation wherein the endoscope needs to be repeatedly inserted into the human body and drawn out from the human body, in order to play the effect of anti-fogging.

1. A device of anti-fogging endoscope system, including a lighting source (1), a transmission fiber (2), and an endoscope (3), wherein the lighting source (1) includes a lighting source of white light (11) and a coupling optical lens (14); the lighting source of white light (11) is a xenon lamp, a white light LED or a mixed white light LED of three primary colors of red, green, and blue, which provides a light output in visible light band; when the xenon lamp is used, the power falls in the range of 250 W˜350 W; when the white light LED is used, the power falls in the range of 50 W˜100 W; when the mixed white light LED of three primary colors of red, green, and blue is used, the power falls in the range of 150 W˜200 W; the coupling optical lens (14) is an optical lens or a set of optical lenses; the transmission fiber (2) is made by binding several fibers having the diameter of several tens of microns together, and is polished on its incident surface and exit surface; the focusing plane of coupling optical lens (14) and the incident surface of transmission fiber (2) are in coincidence; the endoscope (3) includes an endoscope optical lens system (31), a distal optical window plate (32), a light guide fiber bundle (33), an endoscope housing body (34), and a fiber connecting interface (35); the endoscope optical lens system (31) is made of several tens of optical lenses to image the surgical field; the endoscope housing body (34) is mainly made from stainless steel material, which is polished on its surface; the distal optical window plate (32) is a transparent optical material, and can transmit the visible light, which is located in front of the endoscope optical lens system (31), and is welded or adhered to the endoscope housing body (34); the light guide fiber bundle (33) is made by binding several fibers having the diameter of several tens of microns together, and is polished on its incident surface and exit surface; the incident surface of light guide fiber bundle (33) is located inside the fiber connecting interface (35); the incident surface of light guide fiber bundle (33) is connected to the exit surface of transmission fiber (2) in coupling via the fiber connecting interface (35); the light guide fiber bundle (33) is located in the annular space between the optical lens system (31) and the endoscope housing body (34), of which exit surface is against the distal optical window plate (32), which characterized in that, the lighting source (1) also includes a near-infrared lighting source for anti-fogging (12) and a color combination filter (13); the color combination filter (13) is a optical plate, which is located between the lighting source of white light (11) and the coupling optical lens (14) in way of having a intersection angle of 45° from the coupling optical lens (14); the color combination filter (13) is plated with an optical film which transmits the visible light and reflects the near-infrared light; the near-infrared lighting source for anti-fogging (12) is a light source which only emits the near-infrared light, such as a light source of semiconductor laser, a light source of pump laser, and so on, of which the wavelength range of the emitted light is 780 nm˜1100 nm, and the power of the emitted light in full power is between 5 watt and 10 watt; the near-infrared lighting source for anti-fogging (12) is set in way of being perpendicular to the lighting source of white light (11) and having a intersection angle of 45° from the color combination filter (13); the lighting source of white light (11) and the near-infrared lighting source for anti-fogging (12) can be independently controlled to turn on-off and adjust luminance; the distal optical window plate (32) of the endoscope (3) is made from an optical material improving its spectral characteristics, which not only can transmit the visible light, but also can absorb the near-infrared light, wherein the average transmissivity of the visible light is above 80%, and the average absorptivity of the near-infrared light is above 80%.

2. The device of anti-fogging endoscope system according to claim 1, which characterized in that, an anti-fogging method of the anti-fogging endoscope system including the following steps:
(1) 2˜3 minutes before an operation, the near-infrared lighting source for anti-fogging (12) is turned on in full power, near-infrared light transmits through the coupling optical lens (14), the transmission fiber (2), and the light guide fiber bundle (33) in order, and then irradiates on the distal optical window plate (32); the distal optical window plate (32) absorbs energy of the near-infrared light, and the temperature rises up;
(2) After 30 seconds˜1 minute, the temperature of the distal optical window plate rises from room temperature of 25° C. up to about 37° C., then the power of the near-infrared lighting source for anti-fogging (12) is reduced to 10%˜30% of full power to keep the temperature of the distal optical window plate around 37° C.;
(3) Before the doctor inserts the endoscope into a human body, the near-infrared lighting source for anti-fogging (12) is turned off, the lighting source of white light (11) is turned on, and then the endoscope is inserted into the human body; due to the temperature of the distal optical window plate (32) is approximately consistent with the temperature of the human body, no fogging will happen, and so the doctor can perform the normal observation and operation, to achieve the purpose of anti-fogging.

3. The device of anti-fogging endoscope system and its method according to claim 1, which characterized in that, the lighting source of white light (11) is a white light LED having a total power of 80 W; the near-infrared lighting source for anti-fogging (12) is a semiconductor laser having a radiation wavelength of 808 nm and a full power output of 5 W; the distal optical window plate (32) has an absorptivity of 80% at 808 nm, and the average transmissivity of the visible light is 90%; the near-infrared lighting source for anti-fogging (12) is turned on in full power last 1 minute, the temperature of the distal optical window plate (32) rises from room temperature of 25° C. up to about 37° C., then the power is reduced to 30% of full power, keeping the temperature around 37° C.

4. The device of anti-fogging endoscope system and its method according to claim 1, which characterized in that, the lighting source of white light (11) is a mixed white light LED of three primary colors of red, green, and blue having a total power of 170 W; the near-infrared lighting source for anti-fogging (12) is a semiconductor laser having a radiation wavelength of 940 nm and a full power output of 10 W; the distal optical window plate (32) has an absorptivity of 90% at 940 nm, and the average transmissivity of the visible light is 85%; the near-infrared lighting source for anti-fogging (12) is turned on in full power last 30 seconds, the temperature of the distal optical window plate (32) rises from room temperature of 25° C. up to about 37° C., then the power is reduced to 10% of full power, keeping the temperature around 37° C.

5. The device of anti-fogging endoscope system and its method according to claim 1, which characterized in that, the lighting source of white light (11) is a xenon lamp having a total power of 350 W; the near-infrared lighting source for anti-fogging (12) is a Nd:YAG laser having a radiation wavelength of 1064 nm and a full power output of 7 W; the distal optical window plate (32) has an absorptivity of 95% at 1064 nm, and the average transmissivity of the visible light is 80%; the near-infrared lighting source for anti-fogging (12) is turned on in full power last 45 seconds, the temperature of the distal optical window plate (32) rises from room temperature of 25° C. up to about 37° C., then the power is reduced to 20% of full power, keeping the temperature around 37° C.
